(19) United States
(12) Patent Application Publication
(10) Pub. No.: US 2018/0235490 A1
(43) Pub. Date: Aug. 23, 2018

(54) PULSE WAVE DETECTOR

(71) Applicant: KABUSHIKI KAISHA TOSHIBA, Tokyo (JP)

(72) Inventor: Koji UENO, Toda Saitama (JP)

(21) Appl. No.: 15/694,846

(22) Filed: Sep. 3, 2017

(30) Foreign Application Priority Data
Feb. 20, 2017 (JP) 2017-028852

(51) Int. Cl. A61B 5/024; A61B 5/00

(52) U.S. Cl. CPC A61B 5/02427; A61B 5/681

(57) ABSTRACT

According to one embodiment, a pulse wave detector includes a casing connected to a band which together are configured to be worn on a wrist or a forearm of a subject, and at least one light detection unit comprising a light-emitting unit configured to radiate light toward the wrist or the forearm of a subject and a light-receiving unit configured to detect light of the radiated light which has been reflected from the subject, the light-emitting unit and the light-receiving unit spaced from each other in the casing.

### CROSS-REFERENCE TO RELATED APPLICATION

This application is based upon and claims the benefit of priority from Japanese Patent Application No. 2017-028852, filed Feb. 20, 2017, the entire contents of which are incorporated herein by reference.

### FIELD

Embodiments described herein relate generally to a pulse wave detector.

### BACKGROUND

There are pulse wave detectors that radiate light at a human body and detect a pulse waves, which occur as a result of periodic changes in blood flow volume in a body lumen, based on the magnitude of the radiated light which is reflected. In such a pulse wave detector, the precision of the measured pulse waves is preferably high.

### DESCRIPTION OF THE DRAWINGS

FIGS. 1A and 1B are schematic diagrams illustrating a pulse wave detector according to an embodiment.

FIG. 2 is a schematic diagram illustrating the pulse wave detector according to the embodiment.

FIGS. 3A and 3B are schematic diagrams illustrating a pulse wave detector according to a first modification example of the embodiment.

FIGS. 4A and 4B are schematic diagrams illustrating a pulse wave detector according to a second modification example of the embodiment.

### DETAILED DESCRIPTION

An exemplary embodiment provides a pulse wave detector capable of improving the precision of the measured pulse waves.

In general, according to one embodiment, a pulse wave detector includes a casing connected to a band which together are configured to be worn on a wrist or a forearm of a subject, and at least one light detection unit comprising a light-emitting unit configured to radiate light toward the wrist or the forearm of a subject and a light-receiving unit configured to detect light of the radiated light which has been reflected from the subject, the light-emitting unit and the light-receiving unit spaced from each other in the casing.

Hereinafter, exemplary embodiments of the present disclosure will be described with reference to the drawings.

The drawings hereof are schematic and conceptual, and relationships therein between thickness and widths of elements, the ratios of the sizes of elements and portions, and the like may not necessarily be the same as the relationships, ratios, and the like in an actual device. Even for the same elements and portions of a device, the dimensions or ratios of the elements and portions may differ among the drawings in some cases.

In the present specification and the drawings, the same reference numerals are given to the same elements as those previously described and a detailed description thereof will be appropriately omitted.

In each embodiment, “directions” are used. Here, a direction along a forearm from an elbow to a hand of a human body is referred to as a first direction D1 and two directions perpendicular to the first direction D1 and orthogonal to each other are referred to as a second direction D2 and a third direction D3.

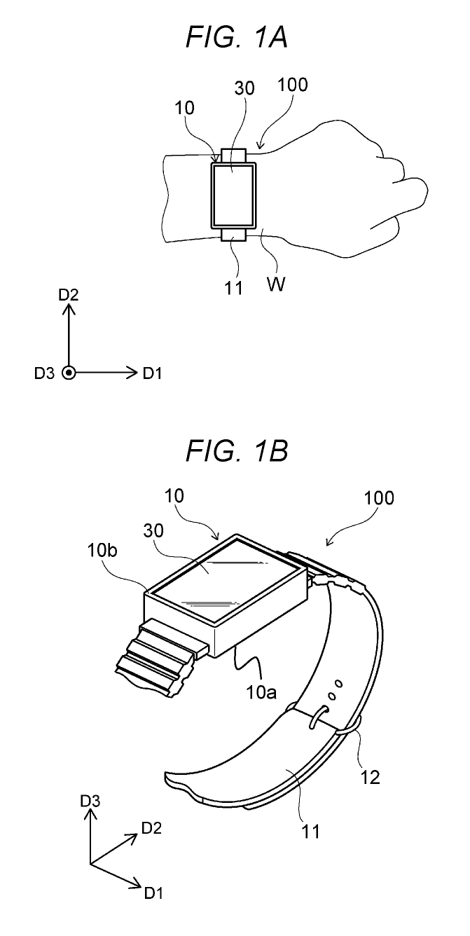

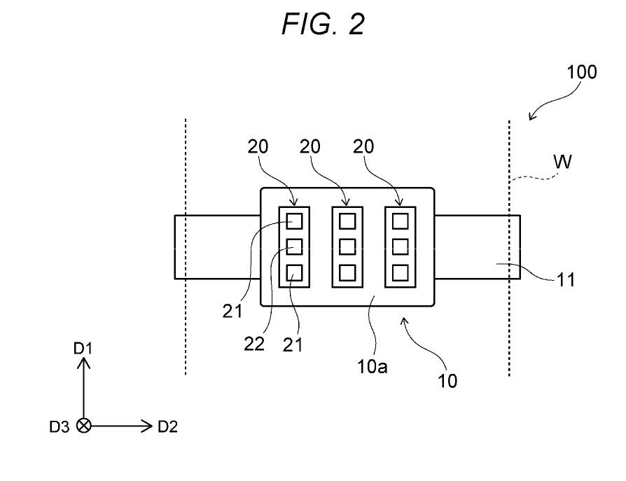

FIGS. 1A, 1B, and 2 are schematic diagrams illustrating a pulse wave detector 100 according to an embodiment.

The pulse wave detector 100 is a wristband type device and is worn on a wrist or a forearm of a target person of which pulse waves are detected. FIGS. 1A, 1B, and 2 illustrate a form in which the pulse wave detector 100 is worn on a wrist W of the human body. FIG. 2 illustrates the pulse wave detector 100 viewed from the side thereof facing the wrist W of the human body.

As illustrated in FIGS. 1A, 1B, and 2, the pulse wave detector 100 includes a casing 10, light detection units 20, and a display unit 30.

The casing 10 includes a belt 11 that is worn on a wrist or a forearm of the human body. In the belt 11, a fastener 12 that fastens the belt 11 together is formed. The casing 10 is configured as, for example, a member with rigidity and the belt 11 is configured with a leather or a resin composition strap with inherent flexibility or as a plurality of consecutively connected metal members.

As illustrated in FIGS. 1B and 2, the casing 10 includes a first surface 10a which comes into contact with a human body at the time of wearing and a second surface 10b on its opposite side. On the first surface 10a, the plurality of light detection unit 20 are provided and are spaced from each other.

Each light detection unit 20 includes a light-emitting unit 21 and a light-receiving unit 22. The light-emitting unit 21 includes a light-emitting element such as a light-emitting diode. The light-receiving unit 22 includes a light-receiving element such as photodiode. In the example illustrated in FIG. 2, the plurality of light-emitting units 21 of each light detection unit 20 are spaced apart from one another in the first direction D1. The light-receiving unit 22 is disposed between the light-emitting units 21.

The light-emitting unit 21 radiates light toward the human body, here the wrist. When the light radiated from the light-emitting unit 21 is reflected by the human body, the reflected light is detected by the light-receiving unit 22. The intensity of the reflected light decreases as the amount of blood flowing in a vessel (artery) of the wrist and the forearm at which the emitted light is directed increases. Therefore, when the light-receiving unit 22 detects then intensity of the light reflected from the human body, a periodic change in the volume of blood flowing through the vessels of the wrist or the forearm can be determined. The pulse wave detector 100 can measure a wearers' pulse rate based on this periodic change in the volume of blood in the vessels over time.

The display unit 30 is disposed on the second surface 10b of the casing 10. The measured pulse rate is displayed on the display unit 30. In addition, a date or a time, various kinds of notifications regarding detection results of the pulse waves, and the like may be displayed on the display unit 30.

The pulse wave detector 100 further includes a control unit and a power supply (battery) (not illustrated) disposed inside the casing 10. The control unit is, for example, a microcomputer and it performs an operation of the light detection unit 20, a process for a detected signal, measurement of a pulse rate, display of information on the display unit 30, and the like based on instructions of the control unit.

Hereinafter, advantages of the embodiment will be described.

When pulse waves are detected, the position of the light detection unit 20 may be distant from the positions of the blood vessels due to a variation in the position of the light detection unit 20 at the time of wearing the pulse wave detector 100, an individual difference in the positions of the blood vessels, or the like. When the blood vessels are distant from the light detection unit 20, the intensity of the signal at the time of detecting reflected light by the light-receiving unit 22 is lowered, and thus the detection precision of the pulse waves deteriorates. In addition, the precision of a pulse rate determination based on the periodic pulses of blood through the vessels also deteriorates.

Accordingly, in the embodiment, as illustrated in FIG. 2, a plurality of light detection units 20 are disposed in the casing 10. Since a plurality of light detection units 20 are provided, any of the light detection units 20 are easily located near a blood vessel. Accordingly, according to the embodiment, the detection precision of the pulse waves by the pulse wave detector 100 is further improved than when only one light detection unit 20 is provided, and thus it is possible to obtain a more accurate pulse rate.

In general, since a thick vessel in which it is easy to detect pulse waves extends in the first direction D1, the light-emitting units 21 and the light-receiving units 22 are preferably arrayed in the first direction D1. Since the light-emitting units 21 and the light-receiving units 22 of each light detection unit 20 are arrayed in the first direction D1, light reflected from vessels are easily incident on the light-receiving units 22, and thus it is possible to increase the intensity of signals detected by the light-receiving units 22. Accordingly, this configuration leads to a further improvement in the detection precision of the pulse waves using the pulse wave detector 100.

The pulse wave detector 100 may use all of the signals detected by the plurality of light detection units 20 or may selectively use one of the signals. For example, the pulse wave detector 100 may measure a pulse rate based on pulse waves obtained by adding and averaging signals detected by the light detection units 20. In addition, the pulse wave detector 100 may measure a pulse rate based on pulse waves obtained by comparing a peak intensity value, an average value of peak intensities, or an average value of signal intensities between signals detected by each of the light detection units 20 and use only a detected signal of the light detection unit 20 having the largest measured value.

For example, as illustrated in FIG. 2, the plurality of light detection units 20 are arrayed across a wrist or a forearm of the human body. Alternatively, the plurality of light detection units 20 may be arrayed in the first direction D1.

The number of light detection units 20 and the numbers of light-emitting units 21 and light-receiving units 22 of each light detection unit 20 are not limited to the example illustrated in FIG. 2 and may be appropriately changed.

### First Modification Example

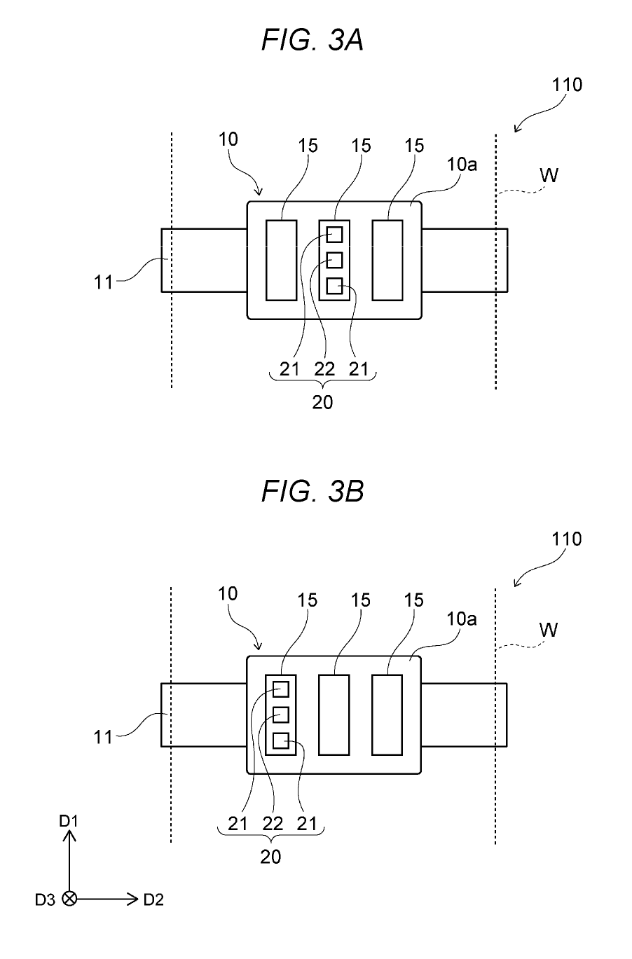

FIGS. 3A and 3B are schematic diagrams illustrating a pulse wave detector 110 according to a first modification example of the embodiment.

FIGS. 3A and 3B illustrate a form when the pulse wave detector 110 is viewed from the side of a wrist W of a human body.

The pulse wave detector 110 is different from the pulse wave detector 100 in that the pulse wave detector 110 includes a plurality of accommodation units 15 and one light detection unit 20 instead of the plurality of light detection units 20.

The casing 10 includes the plurality of accommodation units 15 separated from each other on the first surface 10a. Each accommodation unit 15 is configured to be able to accommodate a light detection unit 20. The light detection unit 20 is detachably provided in any of the accommodation units 15.

For example, a user of the pulse wave detector 110 can detach the light detection unit 20 from one (a first accommodation unit) of the plurality of accommodation units 15 in the state illustrated in FIG. 3A and place the light detection unit 20, or a different light detection unit 20, in a different accommodation unit 15 (a second accommodation unit), as illustrated in FIG. 3B.

That is, the user of the pulse wave detector 110 can change the position of the light detection unit 20 with respect to the casing 10 while confirming a measurement result of a pulse rate. For example, when a measurement result of a pulse rate is considerably different from a normal value or a measurement result of a pulse rate considerably varies for a short time, there is a high possibility of an accurate pulse rate not being obtained. In this case, the user can detach the light detection unit 20 from the accommodation unit 15 and mount the light detection unit 20 in an accommodation unit 15 at a position at which a measurement result of a more accurate pulse rate can be obtained.

In this way, according to the modification example, since the position of the light detection unit 20 with respect to the casing 10 can be changed depending on the measured pulse rate, it is easy to obtain a more accurate pulse rate using the pulse wave detector 110.

When it is determined that an accurate pulse rate is not obtained, the pulse wave detector 110 may display a notification indicating that an accurate pulse rate has not been obtained on the display unit 30 for the user.

### Second Modification Example

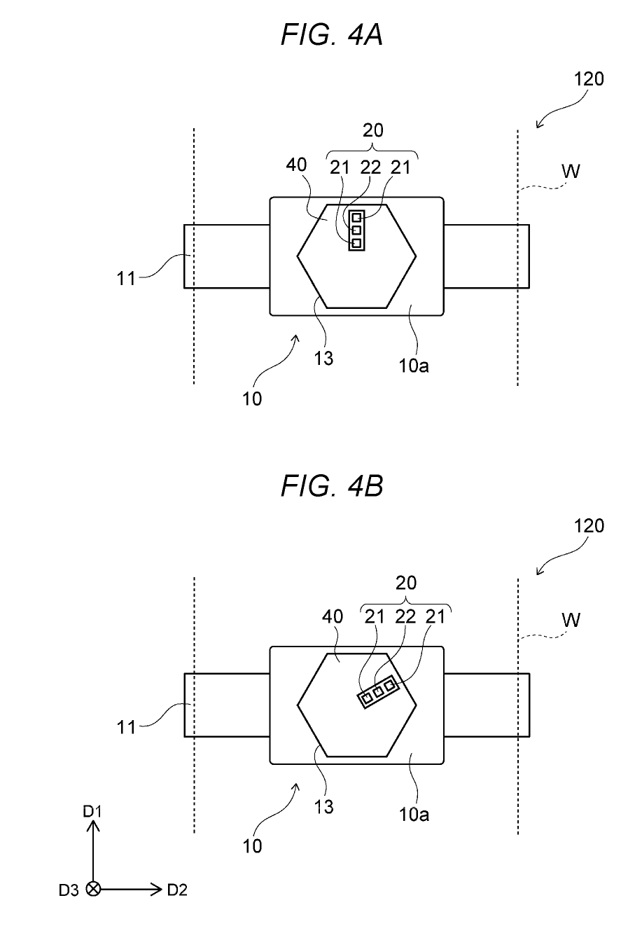

FIGS. 4A and 4B are schematic diagrams illustrating a pulse wave detector 120 according to a second modification example of the embodiment.

FIGS. 4A and 4B illustrate a form when the pulse wave detector 120 is viewed from the side of a wrist W of a human body.

The pulse wave detector 120 is different from the pulse wave detector 100 in that a base 40 on which the light detection unit 20 is located is further included.

In the pulse wave detector 120, the light detection unit 20 is mounted on the base 40. A recess or depression 13 is formed on the first surface 10a of the casing 10. The base 40 is fitted in the depression 13 so that the light detection unit 20 is mounted on the casing 10. The depression 13 has a polygonal perimeter. In the embodiment, the polygonal perimeter of the depression 13, and a matching polygonal perimeter of the base, has sides of equal length.

The base 40 is detachably fitted to the depression 13 of the casing 10. The light detection unit 20 is disposed at a position deviating from the center of the base 40 (the center of the depression 13). Therefore, for example, a user of the pulse wave detector 120 can change the position of the light detection unit 20 with respect to the casing 10 by detaching the base 40 from the state illustrated in FIG. 4A and rotating the base 40 to fit the base 40 in the depression 13, as illustrated in FIG. 4B.

According to the modification example, as in the first modification example, since the position of the light detection unit 20 with respect to the casing 10 can be changed depending on the measured pulse rate, it is easy to obtain a more accurate pulse rate using the pulse wave detector 120.

When the outer edge of the base 40 is circular, there is a possibility of the base 40 being rotated due to a touch to the human body in the state in which the base 40 is fitted in the depression 13. When the base 40 is rotated, the position of the light detection unit 20 with respect to the casing 10 may change. Accordingly, the outer edge of the base 40 (the outer edge of the depression 13) is not circular, but preferably has a shape other than the circular shape, such as hexagonal as illustrated in FIGS. 4A and 4B. Since the shape of the outer edge of the base 40 is not circular, the base 40 is not rotated despite touching of the base 40 to the human body, and thus the position of the light detection unit 20 can be prevented from changing. As a result, it is easy to obtain a more accurate pulse rate using the pulse wave detector 120.

In the pulse wave detector 120 exemplified in FIGS. 4A and 4B, one light detection unit 20 is mounted on the base 40. However, a plurality of light detection units 20 may be disposed on the base 40. Since the positions of the plurality of light detection units 20 with respect to the casing 10 can be changed, it is easier to obtain an accurate pulse rate.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. Specific configurations of the elements such as the casing 10, the light detection unit 20, and the display unit 30 included in the embodiments can be appropriately selected from known technologies by those skilled in the art. The accompanying claims and their equivalents are intended to cover such forms or modifications as would fall within the scope and spirit of the inventions. The above-described embodiments can be combined with each other.

What is claimed is:

1. A pulse wave detector comprising:
a casing connected to a band and together configured to be worn on a wrist or a forearm of a subject; and
at least one light detection unit comprising a light-emitting unit configured to radiate light toward the wrist or the forearm of the subject and a light-receiving unit configured to detect light of the radiated light which has been reflected from the subject, the light-emitting unit and the light-receiving unit spaced from each other in the casing.

2. The pulse wave detector according to claim 1,
wherein in each of the plurality of light detection units, the light-receiving unit and the light-emitting unit are arrayed in a first direction, which, when the pulse wave detector is worn by the subject, extends along the forearm of a human body.

3. The pulse wave detector according to claim 1, wherein the casing comprises a plurality of accommodation units configured to receive a light detection unit therein.

4. The pulse wave detector according to claim 1, wherein a light detection unit is located in only one of the plurality of accommodation units.

5. The pulse wave detector according to claim 1, wherein the casing further comprises a recess extending thereinto, and the light detection unit is connected to a base configured to be received in the recess.

6. The pulse wave detector according to claim 5, wherein the recess has a non-circular perimeter, and the base has a matching non-circular perimeter.

7. The pulse wave detector according to claim 6, wherein the perimeter is a polygon having sides of equal length.

8. A pulse wave detector comprising:
a casing;
a band connected to the casing and configured to attach the casing and band to the wrist or forearm of a human body; and
a light detection unit comprising a light-emitting unit radiating light to the wrist or the forearm of the human body and a light-receiving unit detecting the reflected light of the radiated light, the casing configured such that the position of the light detection unit with respect to the casing is changeable.

9. The pulse wave detector according to claim 8, wherein
the casing includes first and second accommodation units configured to accommodate a light detection unit, the first and second accommodation units are spaced from each other, and
the light detection unit is detachably located in the first or in the second accommodation unit.

10. The pulse wave detector according to claim 8, further comprising:
a base on which the light detection unit is mounted,
wherein the base is detachably fitted into a recess in the casing.

11. The pulse wave detector according to claim 10,
wherein the perimeter of the base and the perimeter of the recess have a shape other than a circular shape.

12. The pulse wave detector according to claim 11, wherein the perimeter of the depression and the perimeter of the base are polygonal, and the sides of the polygon have equal lengths.

13. The pulse wave detector according to claim 12, wherein the orientation of the light detection unit with respect to the casing is changeable.

14. A pulse wave detector, comprising:
a casing;
at least one light detection unit comprising a light-emitting unit configured to radiate light and at least two light-receiving units configured to detect reflected light of the radiated light, the light-emitting unit located between adjacent light detecting units.

15. The pulse wave detector according to claim 14, wherein the casing comprises a plurality of accommodation units configured to receive a light detection unit therein, and only one light detection unit is located in an accommodation unit in the casing.

16. The pulse wave detector according to claim 14, wherein the casing further comprises a recess extending therein, and the light detection unit is connected to a base configured to be received in the recess.

17. The pulse wave detector according to claim 16, wherein the recess has a non circular perimeter, and the base has a matching non-circular perimeter.

18. The pulse wave detector according to claim 17, wherein the perimeter is a polygon having equal side lengths.

19. The pulse wave detector according to claim 18, wherein the orientation of the light detection unit with respect to the casing can be changed.

20. The pulse wave detector according to claim 14, wherein the casing further comprises a display.
